(19) United States
(12) Patent Application Publication
(10) Pub. No.: US 2017/0347240 A1
(43) Pub. Date: Nov. 30, 2017

(54) DETECTING GROUP ACTIVITIES

(71) Applicant: Strava, Inc., San Francisco, CA (US)

(72) Inventors: John Wilde, San Francisco, CA (US); Drew Robb, San Francisco, CA (US)

(21) Appl. No.: 15/167,725

(22) Filed: May 27, 2016

(51) Int. Cl. H04W 4/02; A61B 5/11; A61B 5/00

(52) U.S. Cl. CPC H04W 4/028; A61B 5/7246; A61B 5/1118; A61B 5/1112; A61B 2503/10; A61B 2560/0475

(57) ABSTRACT

Detecting group activities is disclosed, including: receiving a plurality of GPS recorded activities uploaded by a plurality of athletes, wherein the plurality of athletes comprises a first athlete and a second athlete; receiving a request to determine a set of group activities that includes the first athlete and the second athlete; determining the set of group activities based at least in part on a set of time criteria and a set of space criteria used to compare a first GPS recorded activity associated with the first athlete and a second GPS recorded activity associated with the second athlete; and outputting at least some of the set of group activities.

### BACKGROUND OF THE INVENTION

Different athletes may perform physical activities along similar routes. However, athletes' whose physical activities are similar may not be able to easily recognize the similarity of their activities, especially if they are not already acquainted with each other.

### BRIEF DESCRIPTION OF THE DRAWINGS

Various embodiments of the invention are disclosed in the following detailed description and the accompanying drawings.

FIG. 1 is a diagram showing a system for detecting group activities in accordance with some embodiments.

FIG. 2 is a diagram showing an embodiment of a group activity determination server in accordance with some embodiments.

FIG. 3 is a flow diagram showing a process for determining group activities in accordance with some embodiments.

FIG. 4 is a flow diagram showing a process for determining whether two GPS recorded activities form a group activity in accordance with some embodiments.

FIG. 5 is a flow diagram showing a process for determining whether two GPS recorded activities meet time and space criteria associated with forming a group activity in accordance with some embodiments.

FIG. 6 is a flow diagram showing a process for determining an aggregated shared value between a first athlete and a second athlete in accordance with some embodiments.

FIG. 7 is an example of a user interface that presents a first athlete's athletic relationships with other athletes.

FIG. 8 is a flow diagram showing a process for clustering athletes that have been determined to have participated in one or more group activities with a first athlete in accordance with some embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Embodiments of detecting group activities are described herein. A plurality of global positioning system (GPS) recorded activities uploaded by a plurality of athletes is received. In various embodiments, a GPS recorded activity comprises a recorded instance of an athletic performance, such as a run or a bike ride. For example, each GPS recorded activity is recorded by a GPS-enabled device with at least GPS data points and timestamps along various points of the GPS recorded activity. A request to determine a set of group activities that includes a first athlete and a second athlete is received. In various embodiments, a “group activity” that includes a first athlete and a second athlete comprises a pair of GPS recorded activities, in which one GPS recorded activity that is performed by the first athlete is determined to be similar to the other GPS recorded activity that is performed by the second athlete based on a set of time criteria and a set of space criteria. At least some of the set of group GPS recorded activities are output (e.g., sent to and/or presented at a client device).

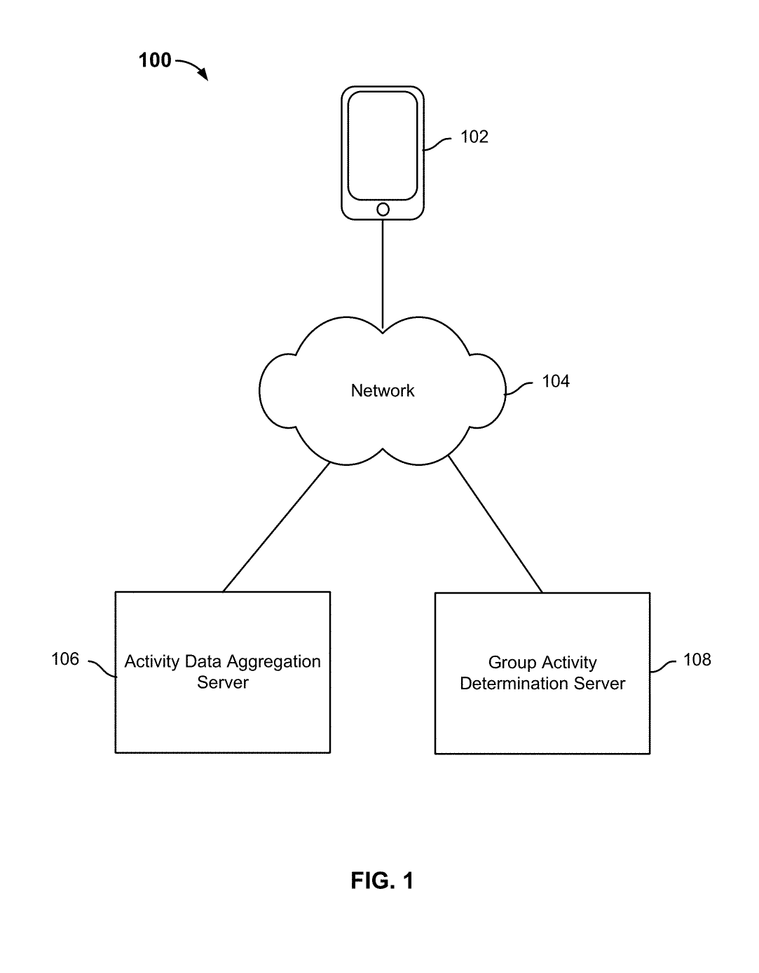

FIG. 1 is a diagram showing a system for detecting group activities in accordance with some embodiments. In the example, system 100 includes device 102, network 104, activity data aggregation server 106, and group activity determination server 108. Network 104 may include high-speed data networks and/or telecommunication networks.

Device 102 is a device that can record GPS data and/or other data associated with a GPS recorded activity. Device 102 can also be a device to which GPS data and/or other data associated with a physical activity can be uploaded or transferred. Examples of device 102 include, but are not limited to: a GPS device (e.g., Garmin Forerunner® and Edge® devices, including Garmin Forerunner® 110, 205, 301, 305, 310XT, 405, 405CX, and Garmin Edge® 305, 605, 705, 500, 800, 810, and 1000), a mobile phone, such as a smart phone (e.g., an Android®-based device or Apple iPhone® device) including a GPS recording application (e.g., MotionX®, Endomondo®, Strava®, and RunKeeper®), a computer, a tablet device, and/or other general purpose computing devices and/or specialized computing devices, which typically include a general processor, a memory or other storage component(s), a network or input/output (I/O) capability, and possibly integrated GPS functionality or support or an interface for a GPS device or GPS functionality.

In various embodiments, device 102 (or an activity tracking application executing thereon) is configured to record GPS data and auxiliary data associated with an athlete activity during the activity. For example, auxiliary data associated with a GPS recorded activity may include physiological, environmental, and/or performance data. In some embodiments, device 102 is configured to receive recorded GPS data and auxiliary data associated with a GPS recorded activity subsequent to the completion of the activity (e.g., such information is uploaded to device 102).

In some embodiments, a “GPS recorded activity” refers to an instance of an athletic performance. Example types of a GPS recorded activity include cycling, running, and skiing. In some embodiments, GPS data includes a series of consecutive and discrete GPS data points (e.g., latitude and longitude coordinates sometimes referred to as “Lat-Lng Data”) with a timestamp for each GPS data point. In some embodiments, auxiliary data includes, but is not limited to, barometric data (e.g., elevation data), heart rate, power/watts (e.g., energy expended), time, speed (e.g., average and/or maximum speed per segment and/or route, in which average speed, for example, can be derived from time and GPS information), and/or cadence. Auxiliary data can be recorded at various granularities. For example, auxiliary data can correspond to each GPS data point, the entire activity (e.g., the auxiliary data includes averages of the metrics), and/or portions of the activity. As an example, one can use device 102 on a bike ride. At the end of the bike ride, the athlete can review his performance with the recorded GPS data (e.g., through a user interface of device 102) to observe the geographical track that he traversed, how much energy he expended along the ride, how fast he finished it in, average speed, elevation-based metrics, and/or other metrics. In some embodiments, device 102 is configured to store the recorded GPS data and the auxiliary data and/or send the recorded data associated with an athlete activity to activity data aggregation server 106. In some embodiments, device 102 is configured to send the recorded data associated with a GPS recorded activity to activity data aggregation server 106 during the GPS recorded activity (e.g., in real-time) and/or after the GPS recorded activity has been completed. In some embodiments, device 102 is configured to present an interactive user interface (e.g., through an activity tracking application executing at device 102). The user interface may display GPS data and receive selections (e.g., made by a user) with respect to the displays. In some embodiments, device 102 sends the selections that it receives to group activity determination server 108.

In some embodiments, a user interface may be presented at device 102. In some embodiments, the user interface may be presented by group activity determination server 108 through an associated activity tracking application executing at device 102 or by another component that is not shown in the example of FIG. 1. In some embodiments, the user interface is configured to receive user inputs such as a confirmation of a suggestion to establish an athletic relationship between a first athlete and a second athlete that is send by group activity determination server 108. The user inputs received at device 102 are configured to be sent group activity determination server 108.

Activity data aggregation server 106 is configured to aggregate recorded athlete activity data from devices such as device 102. In some embodiments, the GPS recorded activity data received at activity data aggregation server 106 is received during the GPS recorded activities (e.g., in real-time) and/or subsequent to the completion of the athletic activities. Activity data aggregation server 106 is configured to store information associated with each GPS recorded activity. For example, information associated with each GPS recorded activity includes an identifier associated with the athlete that performed the activity, the activity type associated with the activity, the date and/or period of time during which the activity took place, the device type that was used to record the activity data, and the equipment used by the athlete during the activity. In some embodiments, the information associated with each GPS recorded activity may include attributes associated with the GPS recorded activity that were input by an athlete and/or attributes associated with the GPS recorded activity that were inferred from the recorded data. In some embodiments, the information associated with each activity is stored by activity data aggregation server 106 in an activity table that comprises an SQL database. Activity data aggregation server 106 is configured to store the set of GPS data (e.g., a set of GPS/Lat-Lng data points) and a corresponding auxiliary data (e.g., barometric/elevation data, timestamps, watts, heart rates, power, etc.) associated with (e.g., recorded GPS data point along) each GPS recorded activity. In some embodiments, the set of GPS data and a corresponding set of auxiliary data associated with each GPS recorded activity are stored by activity data aggregation server 106 in a virtual hard drive (e.g., Amazon Simple Storage Service) associated with dynamically expanding storage availability. Activity data aggregation server 106 is configured to process the data received for each GPS recorded activity and perform spatial indexing for each GPS recorded activity based on the set of GPS data associated with the GPS recorded activity. In some embodiments, in performing spatial indexing, the information from the activity table and the GPS and corresponding auxiliary data from the virtual hard drive are put together and recorded for each recorded GPS data point and put into a PostGIS database or other spatially enabled and indexed data structure. Activity data aggregation server 106 is configured to send the aggregated activity data to group activity determination server 108.

Group activity determination server 108 is configured to determine a set of group activities between a first athlete and a second athlete. In various embodiments, a “group activity” that includes a first athlete and a second athlete comprises a pair of GPS recorded activities, in which one GPS recorded activity that is performed by the first athlete is similar to the other GPS recorded activity that is performed by the second athlete based on a set of time criteria and a set of space criteria. In some embodiments, a group activity may include more than two athletes and comprises a set of GPS recorded activities associated with respective three or more athletes that are similar to each other based on a set of time criteria and a set of space criteria. For purposes of illustration, for the remainder of this application, a group activity will refer to a pair of similar GPS recorded activities that are performed together by a first athlete and a second athlete. Examples of determining whether two GPS recorded activities performed respectively by a first athlete and a second athlete will be described in further detail. At least some group activities that have been determined to include the first athlete and the second athlete can be output for the first athlete and/or the second athlete (e.g., to inform that two athletes of the activities in which they may have, intentionally or inadvertently, performed together) for the first athlete (e.g., at a device used by the first athlete such as device 102). In some embodiments, at least some athletes with whom the first athlete is determined to have performed group activities with are output for the first athlete (e.g., at a device used by the first athlete such as device 102).

In some embodiments, group activity determination server 108 is configured to initiate a determination of a set of group activities that includes a first athlete and a second athlete in response to a trigger event. For example, the trigger event comprises receiving a newly uploaded GPS recorded activity associated with the first athlete from device 102 or activity data aggregation server 106. Group activity determination server 108 can proceed to make pairwise comparisons between each of at least a subset of the GPS recorded activities that are associated with the first athlete (including the newly uploaded GPS recorded activity) and each of at least a subset of historical GPS recorded activities associated with a second athlete. For example, the GPS recorded activities associated with the first athlete and the second athlete may be obtained from activity data aggregation server 106.

In some embodiments, group activity determination server 108 is configured to determine aggregate shared values from a set of group activities that includes a first athlete and a second athlete. In various embodiments, a “shared value” comprises an attribute value that describes a particular group activity. In some embodiments, a “shared value” comprises an attribute that is common to both of the GPS recorded activities that are included in a group activity. In some embodiments, a shared value may be numerical such as, for example, the time the two athletes spent together during a group activity, a number of stops during a group activity, a distance traveled together during a group activity, and an average speed during a group activity. In some embodiments, a shared value may be non-numerical such as, for example, a starting location of a group activity, an ending location of a group activity, an activity type (e.g., biking, running) associated with a group activity, one or more stops (e.g., and venues associated with the stops during a group activity), photos taking during a group activity, a device used to record a group activity, and a type of equipment used using a group activity (e.g., a type of bike used for a biking activity, a type of shoe used during a running activity). In some embodiments, the shared value associated with each group activity that includes a first athlete and a second athlete are aggregated to determine an aggregated shared value associated with the entire set of group activities associated with the first athlete and the second athlete. In some embodiments, aggregating shared values of the same type associated with at least a subset of group activities that include the first athlete and the second athlete into an aggregate shared value includes determining the union of the at least subset of shared values (e.g., determining a set of all unique shared values across the set of group activities), combining (e.g., adding) together the at least subset of shared values, finding the most common value from the at least subset of, and/or finding an average (e.g., a mean or median) of the at least subset of shared values. One or more shared values can be aggregated across the entire set of group activities associated with the first athlete and the second athlete. In some embodiments, an aggregated shared value between a first athlete and a second athlete is compared against a predetermined threshold aggregated shared value and in the event that the aggregated shared value exceeds the predetermined threshold aggregated shared value, an athletic relationship may be suggested to be established between the first athlete and the second athlete. For example, if the aggregated shared value comprises a total amount of time that two athletes performed group activities together exceeds a corresponding predetermined total group activity threshold time, then the first athlete may be suggested to establish an athletic relationship (e.g., a friendship, a following relationship, a training partner status) that is recognized by a platform associated with determining group activities with the second athlete. In some embodiments, group activity determination server 108 is configured to share (e.g., anonymized) established athletic relationship(s) between the first athlete and one or more other athletes at a third party social network profile belonging to a first athlete by sending data associated with such relationships to a social network server (not shown in the diagram). In some embodiments, group activity determination server 108 is configured to compare established athletic relationship(s) between the first athlete and one or more other athletes to other known relationships between the first athlete and other athletes that are derived from third party sources (e.g., third part social network networks that are not shown in the diagram) or processes (e.g., whether or not athletes are connected in an existing “friend” or “following” relationship on a social network).

In some embodiments, various other athletes with which a first athlete is determined to have performed group activities can be clustered into one or more clusters based on attribute values associated with the various other athletes. In some embodiments, various attributes that are stored for the athletes for which each has been determined to each have performed at least one group activity with the first athlete, are used to cluster these athletes. For example, if athlete Alice is determined to have performed group running activities with each of Bonnie, Carrie, and Dana, and both Bonnie and Carrie are determined to have run with Alice during weekend morning, then Bonnie and Carrie can be added to a “Weekend Runners” cluster.

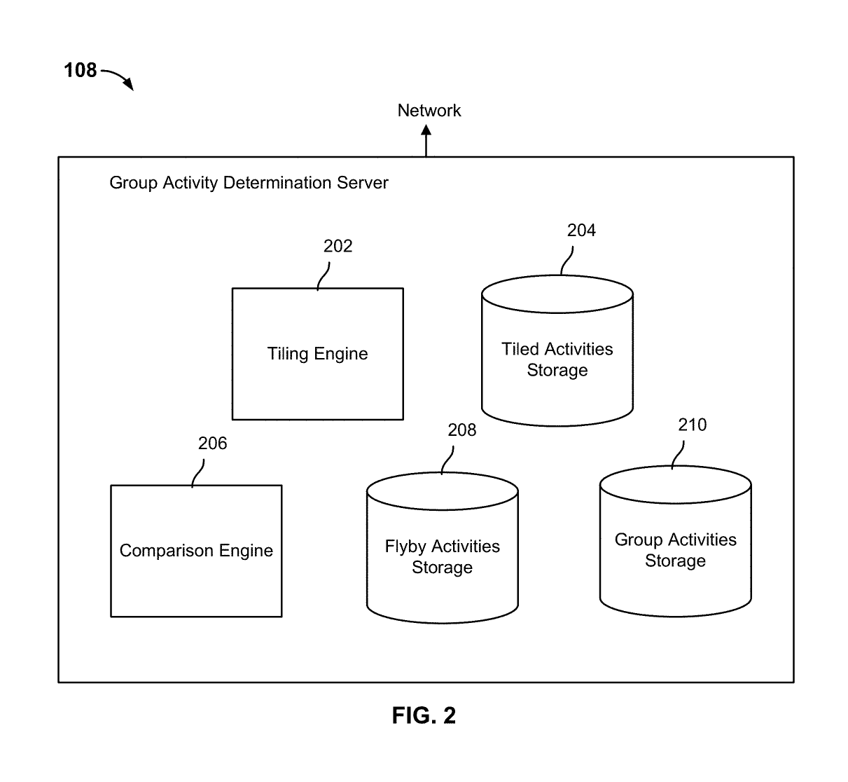

FIG. 2 is a diagram showing an embodiment of a group activity determination server in accordance with some embodiments. In some embodiments, group activity determination server 108 of system 100 of FIG. 1 can be implemented with the example of FIG. 2. In the example, the group activity determination server comprises tiling engine 202, tiled activities storage 204, comparison engine 206, flyby activities storage 208, and group activities storage 210. Each of tiling engine 202 and comparison engine 206 can be implemented, for example, as distinct or integrated software components, which can include module(s), package(s), and/or other distinct or integrated sub-components to provide an executable computer program that can perform these described functions when executed on a processor, and can be implemented using a programming language such as Go, Java, Python, Objective C, and/or other programming languages. An example hardware computing environment to execute the components of FIG. 2 includes a cloud computing service, such as Amazon's Web Services. Each of tiled activities storage 204, flyby activities storage 208, and group activities storage 210 may be implemented as one or more databases (e.g., MySQL or Apache Cassandra databases) that may be running Apache Cassandra.

Tiling engine 202 is configured to determine a set of tiles to which each GPS recorded activity maps. In various embodiments, a “tile” comprises a rectangular geographical area (e.g., that is 20 meters by 20 meters). For example, determining a set of tiles to which a GPS recorded activity maps includes adding any tile that includes a GPS data point from a GPS recorded activity (and also, in some embodiments, any tiles in between two such tiles) in the set of tiles. Tiling engine 202 is configured to store each set of tiles associated with identifying information associated with the corresponding GPS recorded activity in tiled activities storage 204.

Comparison engine 206 is configured to perform pairwise comparisons between sets of tiles associated with two different GPS recorded activities. In some embodiments, the sets of tiles compared by comparison engine 206 are stored in tiled activities storage 204. In various embodiments, comparison engine 206 is configured to compare a first set of tiles associated with a GPS recorded activity performed by a first athlete with a second set of tiles associated with a GPS recorded activity performed by a second athlete to determine whether they meet a set of time criteria and also a set of space criteria to qualify as a group activity between the first athlete and a second athlete. As will be described in further detail below, one example technique of determining whether a first set of tiles and a second set of tiles meet a set of time criteria and also a set of space criteria to qualify as a group activity between the first athlete and a second athlete is as follows: First, for each tile in a set of tiles associated with a given GPS recorded activity, comparison engine 206 is configured to query tiled activities storage 204 for every GPS recorded activity that includes a GPS data point in that tile (e.g., and/or its eight immediate neighboring tiles) within a predetermined time window (e.g., two minutes) of a timestamp associated with the GPS data point of the given GPS recorded activity in that tile. The union (e.g., the unique/distinct elements) of all GPS recorded activities that are associated with respective sets of tiles that share at least one tile (or include a tile that is one of eight immediate neighboring tiles) within the predetermined time window with the set of tiles associated with the given GPS recorded activity comprises a set of candidate activities relative to the given GPS recorded activity. In some embodiments, each candidate activity relative to the given GPS recorded activity represents a GPS recorded activity that was proximate to the given GPS recorded activity for a least a short period of the GPS recorded activity and is sometimes referred to as a “flyby activity” relative to the given GPS recorded activity. Comparison engine 206 can store data associated with each determined flyby activity relative to the given GPS recorded activity in flyby activities storage 208. For example, data associated with each determined flyby activity relative to the given GPS recorded activity may include identifiers associated with the pair of activities and identifiers associated with the pair of athletes that had performed the two respective activities. In some embodiments, flyby activities and/or the athletes that performed them relative to a given GPS recorded activity are output (e.g., at a client device associated with the athlete associated with the give GPS recorded activity). Next, comparison engine 206 is configured to determine a distance (space) correlation between the given GPS recorded activity and each of the candidate activities in the set. For example, a dynamic time warping algorithm can be applied to the pair of GPS recorded activities (that includes the given GPS recorded activity and a candidate activity) to determine a mapping that that includes for each of at least some GPS data points in the given GPS recorded activity, a corresponding (e.g., closest in timestamps) GPS data point in the candidate activity. A pointwise correlation (e.g., a Gaussian-based) function is computed for each pair of GPS data points in this mapping. The correlation values are then weighted by the distance to adjacent GPS data points in each GPS recorded activity. Finally, a sum is taken and then normalized by dividing by the total length of both GPS recorded activities to determine an overall distance correlation between the given GPS recorded activity and the candidate activity. In the event that the distance correlation between the given GPS recorded activity and the candidate activity exceeds a predetermined correlation threshold (e.g., 0.3), then the given GPS recorded activity and the candidate activity are considered to form a group activity between the two athletes that respectively performed the given GPS recorded activity and the candidate activity.

Comparison engine 206 is configured to store data associated with each determined group activity in group activities storage 210. For example, data associated with each determined group activity that includes a first GPS recorded activity and a second GPS recorded activity may include identifiers associated with the pair of activities, identifiers associated with the pair of athletes that had performed the two respective activities, and the computed distance correlation between the pair of activities.

Comparison engine 206 is configured to perform searches (e.g., input by a first athlete) among group activities that include the first athlete. For example, comparison engine 206 can receive a search query submitted by a first athlete and compare the search query to the set of group activities and/or other athletes associated with the set of group activities that include the first athlete and one or more other athletes. Then, comparison engine 206 can output (e.g., send to and/or present at a client device used by the first athlete) the one or more group activities and/or the one or more other athletes associated with the set of group activities that match the search query. For example, a first athlete may submit a search query that includes an identifier associated with another athlete to comparison engine 206 and comparison engine 206 can determine whether the identified other athlete has performed a group activity with the first athlete and/or which group activities that the identified athlete has performed with the first athlete.

In various embodiments, comparison engine 206 is configured to determine an aggregate shared value associated with group activities that include a first athlete and a second athlete. In various embodiments, a “shared value” comprises a common value associated with a group activity that includes a pair of GPS recorded activities that have been performed by two athletes. In various embodiments, a shared value can be a numerical attribute or a non-numerical attribute. For example, a numerical shared value associated with a group activity between a first athlete and a second athlete includes the total distance traveled together by the first and second athletes during the group activity, the average speed associated with the group activity, and the total time traveled together by the first and second athletes during the group activity. For example, the total distance traveled together by the first and second athletes during the group activity can be estimated by multiplying the total distance of the GPS recorded activity for either of the first athlete or the second athlete by the distance correlation (e.g., a value on a scale of 0 to 1 or a percentage value). For example, a non-numerical shared value associated with a group activity between a first athlete and a second athlete includes the starting location associated with the group activity, the ending location associated with the group activity, the activity type associated with the group activity, the places visited during group activity, the popular visited places associated with the group activity (including starting and/or ending locations), the device used to record the pair of activities associated with the group activity, the type of equipment used by the pair of activities associated with the group activity. Comparison engine 206 can aggregate all the shared values of the same type associated with at least a subset of group activities that include the first athlete and the second athlete. In various embodiments, “aggregating” shared values of the same type associated with at least a subset of group activities that include the first athlete and the second athlete into an aggregate shared value includes determining the union of the at least subset of shared values (e.g., determining a set of all unique shared values across the set of group activities), combining (e.g., adding) together the at least subset of shared values, finding the most common value from the at least subset of, and/or finding an average (e.g., a mean or median) of the at least subset of shared values. In some embodiments, comparison engine 206 is configured to compare an aggregate shared value associated with a set of group activities including a first athlete and a second athlete to a predetermined threshold aggregate shared value and if the aggregate shared value exceeds the predetermined threshold aggregate shared value, then comparison engine 206 is configured to suggest an athletic relationship to be established between the first athlete and the second athlete. For example, if the aggregated shared value comprises a total amount of time that two athletes performed group activities together exceeds a corresponding predetermined total group activity threshold time, then the first athlete may be suggested to establish an athletic relationship (e.g., a friendship, a following relationship, a training partner status), which is recognized by a platform associated with determining group activities, with the second athlete. In some embodiments, comparison engine 206 is configured to share (e.g., anonymized) established athletic relationship between the first athlete and the second athlete at a third party social network profile belonging to a first athlete by sending data associated with such relationships to a social network server (not shown in the diagram). In some embodiments, comparison engine 206 is configured to compare established athletic relationships between the first athlete and the second athlete to other known relationships between the first athlete and the second athlete that are derived from third party sources (e.g., not shown in the diagram) or processes (e.g., whether or not athletes are connected in an existing “friend” or “following” relationship on a social network). For example, if it is determined that an athletic relationship had been established between the first athlete and the second athlete but that the first athlete and the second athlete does not have an established relationship at a social network, then a suggestion for a relationship to be established between the first athlete and the second athlete at the social network can be sent by comparison engine 206 to a server associated with the social network.

In some embodiments, comparison engine 206 is configured to cause a set of GPS recorded activities performed by various athletes that have been determined to be included in various group activities with a given athlete to be presented for that given athlete (e.g., at a client device used that the given athlete). For example, comparison engine 206 can send, to a client device associated with a first athlete, data associated with GPS recorded activities performed by athletes other than the first athlete that have been determined to be included in group activities with GPS recorded activities that have been performed by the first athlete. This way, the first athlete can be made aware of all the GPS recorded activities with which the first athlete had intentionally or unintentionally performed group activities.

In some embodiments, comparison engine 206 is configured to cluster athletes of a set of GPS recorded activities that have been determined to be included in group activities with a first athlete. For example, comparison engine 206 is configured to compare the athletes that have been determined to have performed group activities with the first athlete based on attributes associated with the athletes and/or from their GPS recorded activities that have been determined to be included in group activities with GPS recorded activities performed by the first athlete. For example, the set of athletes can be clustered into groups such as “racers”, “morning riders”, “commuters”, “swimmers”, “runners”, “multisport athletes” such as “triathletes”, “coworkers”, etc. In some embodiments, comparison engine 206 is configured to cause the clusters of athletes to be presented at a client device associated with the first athlete.

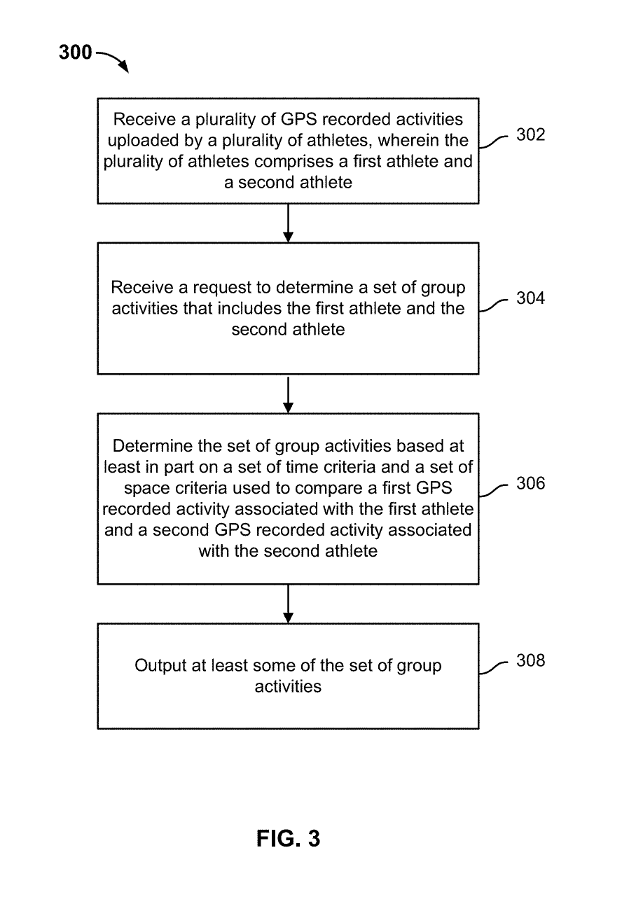

FIG. 3 is a flow diagram showing a process for determining group activities in accordance with some embodiments. In some embodiments, process 300 is implemented at a system such as system 100 of FIG. 1.

At 302, a plurality of GPS recorded activities uploaded by a plurality of athletes is received, wherein the plurality of athletes comprises a first athlete and a second athlete. GPS recorded activities performed by various athletes are uploaded. In various embodiments, the GPS recorded activities are associated with the same type of athletic activity (e.g., cycling or running).

At 304, a request to determine a set of group activities that includes the first athlete and the second athlete is received. For example, the request to determine group activities that includes a first and second athlete is automatically issued when either one of the first or second athlete uploads a new GPS recorded activity.

At 306, the set of group activities is determined based at least in part on a set of time criteria and a set of space criteria used to compare a first GPS recorded activity associated with the first athlete and a second GPS recorded activity associated with the second athlete. In various embodiments, pairwise comparisons are made between each of at least a subset of GPS recorded activities that were uploaded by the first athlete and each of at least a subset of GPS recorded activities that were uploaded by the second athlete. Pairs of a GPS recorded activity performed by a first athlete and a GPS recorded activity performed by a second athlete that meet a set of time criteria and a set of space criteria are determined to be sufficiently correlated (e.g., sufficiently correlated in space and are sufficiently contemporaneous) and therefore form a group activity that is performed by the first athlete and the second athlete.

At 308, at least some of the set of group activities is output. In some embodiments, at least some of the group activities that are performed by the first athlete and the second athlete are presented at the client device of one or both of the first athlete and the second athlete.

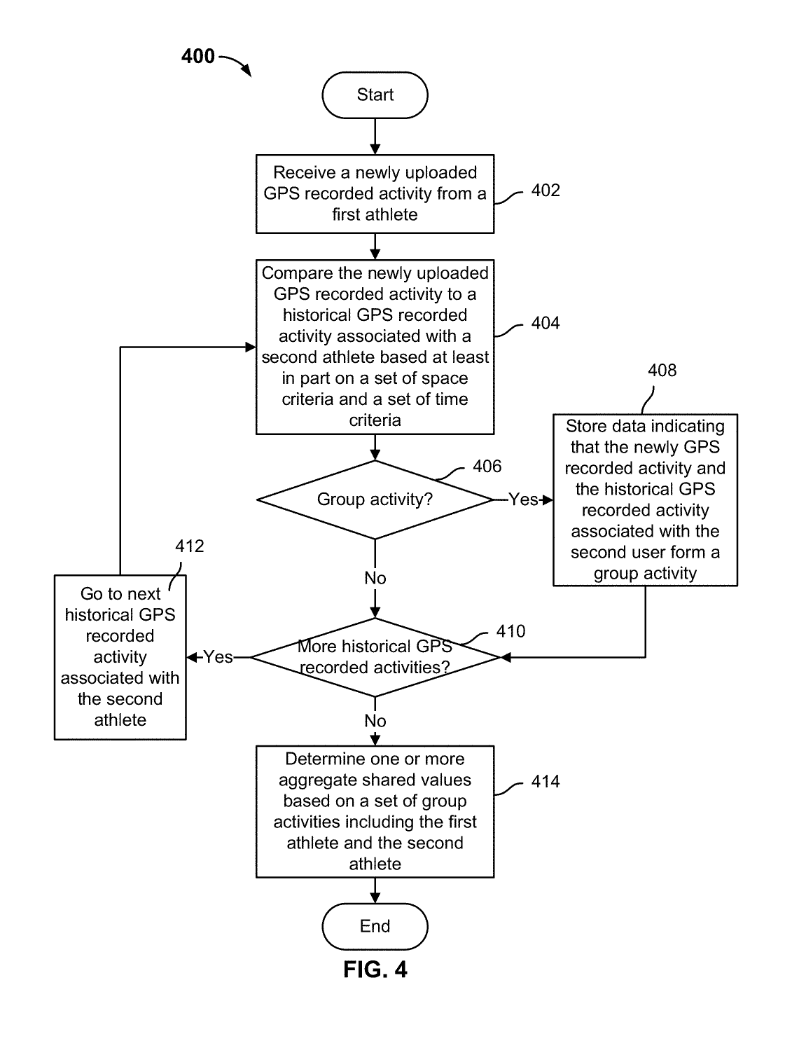

FIG. 4 is a flow diagram showing a process for determining whether two GPS recorded activities form a group activity in accordance with some embodiments. In some embodiments, process 400 is implemented at a system such as system 100 of FIG. 1. In some embodiments, steps 304 and 306 of process 300 of FIG. 3 can be implemented using the example of process 400.

Process 400 describes an example of a process of comparing a newly uploaded GPS recorded activity uploaded from a first athlete to historical GPS recorded activities associated with a second athlete. Process 400 can be extended to compare one or more historical GPS recorded activities associated with the first athlete with those of one or more other athletes. A historical GPS recorded activity associated with an athlete refers to a GPS recorded activity that was previously uploaded by that athlete.

At 402, a newly uploaded GPS recorded activity is received from a first athlete. In some embodiments, a trigger event is configured to cause comparisons between GPS recorded activities associated with one athlete to be compared with the GPS recorded activities associated with one or more other athletes. In the example of process 400, such a trigger event is the uploading of a new GPS recorded activity by a first athlete. For example, the first athlete has recently completed recording a new physical activity and decided to upload it to the server.

At 404, the newly uploaded GPS recorded activity is compared to a historical GPS recorded activity associated with a second athlete based at least in part on a set of space criteria and a set of time criteria. A pairwise comparison is made between the newly uploaded GPS recorded activity associated with the first athlete and a historical GPS recorded activity associated with the second athlete. In some embodiments, the set of space criteria and the set of time criteria are predetermined.

At 406, it is determined whether the newly uploaded GPS recorded activity associated with the first athlete and the historical GPS recorded activity associated with the second athlete form a group activity based at least in part on the comparison. In the event that it is determined that the newly uploaded GPS recorded activity associated with the first athlete and the historical GPS recorded activity associated with the second athlete form a group activity, control is transferred to 408. Otherwise, in the event that newly uploaded GPS recorded activity associated with the first athlete and the historical GPS recorded activity associated with the second user do not form a group activity, control is transferred to 410. If it is determined that the newly uploaded GPS recorded activity associated with the first athlete and the historical GPS recorded activity associated with the second athlete meet both the set of space criteria and the set of time criteria, then it is determined that the two GPS recorded activities are sufficiently contemporaneous in time and also sufficiently correlative in space with respect to each other to be considered a group activity. Therefore, the two sets of criteria can determine whether two GPS recorded activities are close enough in both time and space such that the users that performed the two respective activities had performed a group activity.

At 408, data indicating that the newly uploaded GPS recorded activity and the historical GPS recorded activity form a group activity is stored. When two GPS recorded activities are determined to form a group activity, then the group activity relationship between the two activities is stored.

At 410, it is determined whether there is at least one more historical GPS recorded activity associated with the second athlete. In the event that the second athlete is associated with at least one more GPS recorded activity, control is transferred to 412, at which process 400 starts again from step 404 on the next historical GPS recorded activity associated with the second athlete. Otherwise, in the event that the second athlete is not associated with at least one more GPS recorded activity, control is transferred to 414. The newly uploaded GPS recorded activity is compared with each historical GPS recorded activity associated with the second user to determine whether the newly uploaded GPS recorded activity forms a group activity with any of them.

At 414, one or more aggregate shared values are determined based on a set of group activities including the first athlete and the second athlete. A set of all determined group activities that have been determined to include both the first athlete and the second athlete (e.g., group activities that includes the newly uploaded GPS recorded activity by the first athlete as well as all historical GPS recorded activity by the first and second athletes) is determined and one or more types of shared values can be determined for each such group activity. Then, shared values of the same type across the entire set of group activities that include the first and second athletes can be aggregated to determine respective aggregate shared values. In some embodiments, one or more of the aggregate shared values can be presented to one or both of the athletes and/or compared against respective predetermined threshold values (e.g., such that if a threshold value is exceeded, a predetermined action can be performed).

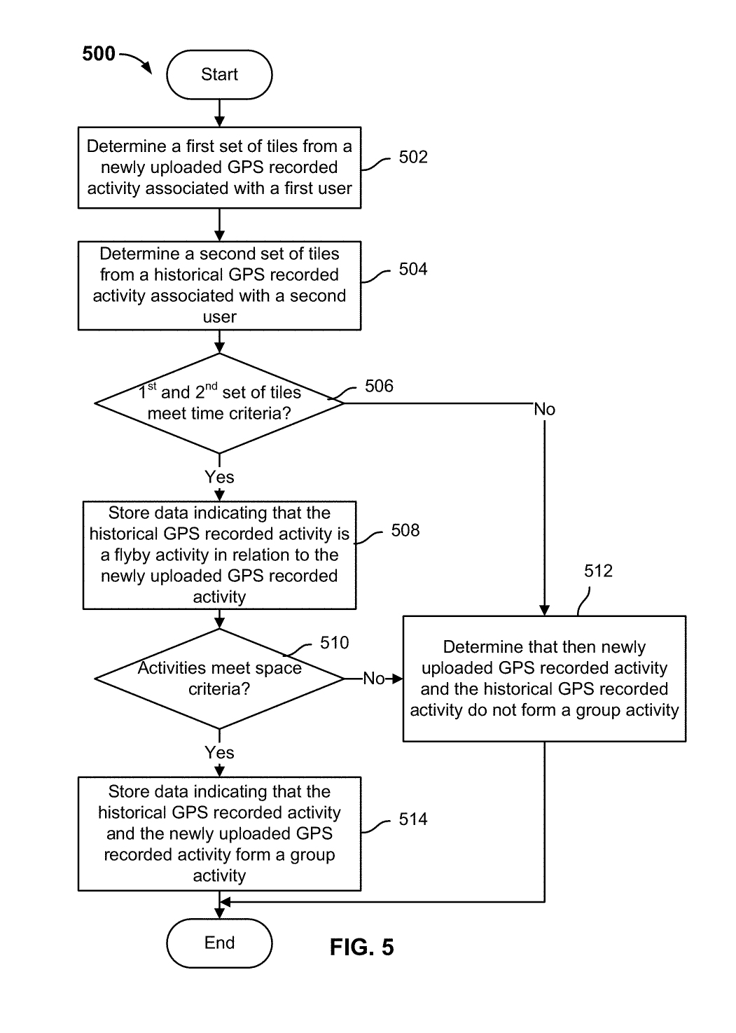

FIG. 5 is a flow diagram showing a process for determining whether two GPS recorded activities meet time and space criteria associated with forming a group activity in accordance with some embodiments. In some embodiments, process 500 is implemented at a system such as system 100 of FIG. 1. In some embodiments, steps 404 and 406 of process 400 of FIG. 4 can be implemented using the example of process 500.

Process 500 describes determining whether a newly uploaded GPS recorded activity uploaded from a first athlete and a historical GPS recorded activity associated with a second athlete meet specific examples of time criteria and space criteria.

At 502, a first set of tiles is determined from a newly uploaded GPS recorded activity associated with a first athlete. The newly uploaded GPS recorded activity is converted into a set of geographical ties. As described above, a tile (and one or more neighboring tile thereof) that includes a GPS data point of a GPS recorded activity is included in the converted set of tiles.

At 504, a second set of tiles is determined from a historical GPS recorded activity associated with a second athlete. The historical GPS recorded activity is also converted into a set of geographical ties.

At 506, it is determined whether the first set of tiles and a second set of tiles meet a set of time criteria. In the event that the first set of tiles and the second set of tiles meet the set of time criteria, control is transferred to 508. Otherwise, in the event that the first set of tiles and the second set of tiles do not meet the set of time criteria, control is transferred to 512 and the newly uploaded GPS recorded activity uploaded from the first athlete and the historical GPS recorded activities associated with the second athlete do not form a group activity. One example set of time criteria involves comparing the two sets of tiles to determine whether for at least one tile in the first set of tiles, the second set of tiles includes the same tile (or a neighboring tile thereof) that includes a GPS data point that is associated with a timestamp that is within a predetermined window of time of the timestamp associated with the GPS data point of the first set of tiles in that tile. For example, the predetermined window of time between GPS data points included in the two respective GPS recorded activities is two minutes.

At 508, data indicating that the historical GPS recorded activity is a flyby activity in relation to the newly uploaded GPS recorded activity is stored. If the set of time criteria is met (e.g., the two sets of tiles share at least one tile or include neighboring tiles that include GPS data points that were recorded within a predetermined window of time of each other), then it can be determined that one of the activities at least comprises a flyby activity (e.g., activities whose path cross at least one location) to the other and a flyby relationship between the two activities can be stored. In some embodiments, flyby activities can be stored for the first athlete and/or outputted for (e.g., sent to and/or presented at a client device of) the first athlete.

At 510, it is determined whether the newly uploaded GPS recorded activity uploaded from the first athlete and the historical GPS recorded activities associated with the second athlete meet a set of space criteria. In the event that the newly uploaded GPS recorded activity uploaded from the first athlete and the historical GPS recorded activities associated with the second athlete meet the set of space criteria, control is transferred to 514. Otherwise, in the event that the newly uploaded GPS recorded activity uploaded from the first athlete and the historical GPS recorded activities associated with the second athlete do not meet the set of space criteria, control is transferred to 512 and the newly uploaded GPS recorded activity uploaded from the first athlete and the historical GPS recorded activity associated with the second athlete do not form a group activity. One example of determining whether the two activities meet a set of space criteria is the following: A dynamic time warping algorithm can be applied to the pair of GPS recorded activities to determine a mapping that that includes for each of at least some GPS data points in the newly uploaded GPS recorded activity, a corresponding (e.g., closest in timestamps) GPS data point in the historical GPS recorded activity. A pointwise correlation (e.g., a Gaussian-based) function is computed for each pair of GPS data points in this mapping. The correlation values are then weighted by the distance to adjacent GPS data points in each GPS recorded activity. Finally, a sum is taken and then normalized by dividing by the total length of both GPS recorded activities to determine an overall distance correlation between the newly uploaded GPS recorded activity and the historical GPS recorded activity. In the event that the distance correlation between the newly uploaded GPS recorded activity and the historical GPS recorded activity exceeds a predetermined correlation threshold (e.g., 0.3 on a scale of 0 to 1, where 0 represents the lowest/no correlation and 1 represents the greatest correlation), then the newly uploaded GPS recorded activity and the historical GPS recorded activity are considered to form a group activity between the two athletes that respectively performed the newly uploaded GPS recorded activity and the historical GPS recorded activity.

At 514, data indicating that the newly uploaded GPS recorded activity uploaded from the first athlete and the historical GPS recorded activities associated with the second athlete form a group activity is stored.

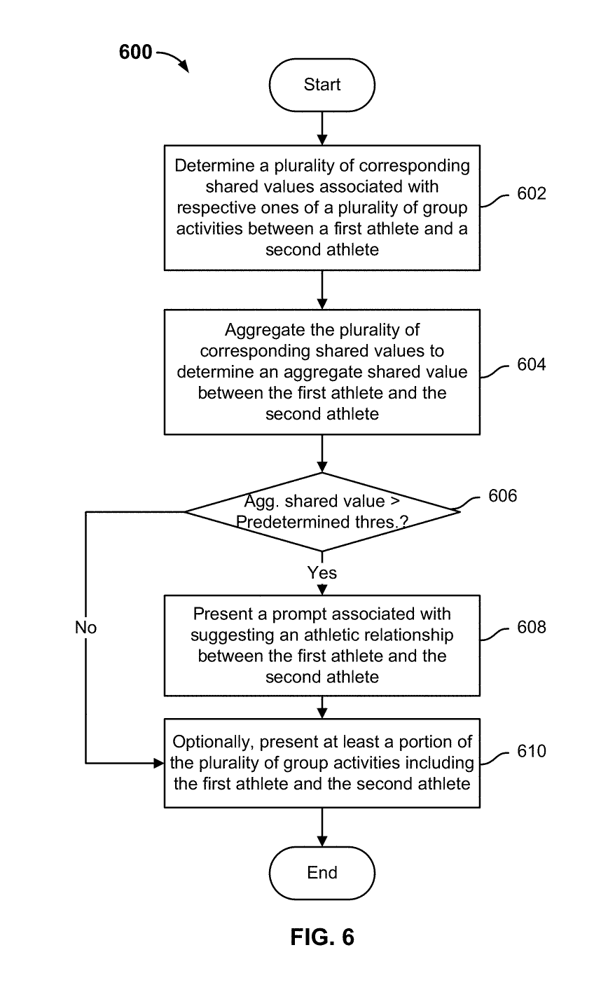

FIG. 6 is a flow diagram showing a process for determining an aggregated shared value between a first athlete and a second athlete in accordance with some embodiments. In some embodiments, process 600 is implemented at a system such as system 100 of FIG. 1. In some embodiments, step 414 of process 400 of FIG. 4 can be implemented using the example of process 600.

Process 600 describes determining an aggregated shared value between a first athlete and a second athlete based on a set of group activities that have been determined to include both the first athlete and the second athlete.

At 602, a plurality of corresponding shared values associated with respective ones of a plurality of group activities between a first athlete and a second athlete is determined. In some embodiments, after one or more group activities that include a first athlete and a second athlete have been determined, shared values associated with each such group activity can be determined. As mentioned above, in some embodiments, a “shared value” comprises an attribute that is common to both of the GPS recorded activities that are included in a group activity. Thus, one or more types of shared values can be determined for each group activity that includes a pair of activities that were performed by the first athlete and the second athlete. In some embodiments, a shared value may be numerical such as, for example, the time the two athletes spent together during a group activity, a number of stops during a group activity, a distance traveled together during a group activity, and an average speed during a group activity. In some embodiments, a shared value may be non-numerical such as, for example, a starting location of a group activity, an ending location of a group activity, an activity type (e.g., biking, running) associated with a group activity, one or more stops (e.g., and venues associated with the stops during a group activity), photos taking during a group activity, a device used to record a group activity, and a type of equipment used using a group activity (e.g., a type of bike used for a biking activity, a type of shoe used during a running activity).

At 604, the plurality of corresponding shared values is aggregated to determine an aggregated shared value between the first athlete and the second athlete. Shared values associated with the group activities that are of the same type can be aggregated into an aggregated shared value that is associated with the pair including the first athlete and the second athlete. In some embodiments, aggregating shared values includes adding/combining together the shared values. For example, the time traveled together associated with each group activity can be added together to determine the first and second athlete's aggregate time traveled together across their group activities.

At 606, it is determined whether the aggregated shared value is greater than a predetermined threshold aggregated shared value. In the event that it is determined that the aggregated shared value is greater than a predetermined threshold aggregated shared value, control is transferred to 608. Otherwise, in the event that it is determined that the aggregated shared value is not greater than a predetermined threshold aggregated shared value, control is transferred to 610. In some embodiments, where a type of aggregated shared value comprises a numerical value, it can be compared to a predetermined threshold aggregated shared value associated with the same type (e.g., different predetermined threshold aggregated shared values can be stored for different types of aggregated shared values).

At 608, a prompt associated with suggesting an athletic relationship between the first athlete and the second athlete is presented. If the aggregated shared value is greater than a predetermined threshold aggregated shared value, then an athlete relationship between the first athlete and the second athlete can be sent to and presented at a client device used by the first athlete or a client device used by the second athlete. The prompt can include a control that once selected, establishes an athletic relationship between the first athlete and the second athlete at the server. For example, the athletic relationship may comprise a “training partner,” a friendship, or a following relationship.

In some embodiments, prior to sending the prompt to either one of the first athlete and the second athlete, whether an athletic relationship already exists between the first user and the second user can be first determined. Then, the prompt associated with suggesting an athletic relationship between the first athlete and the second athlete may only be sent to either of the first athlete and the second athlete in the event that such a relationship is not already established between the two athletes.

At 610, optionally, at least a portion of the plurality of group activities including the first athlete and the second athlete are presented. Information associated at least some of the group activities that include the first and second athletes are sent to and presented at a client device used by the first athlete or a client device used by the second athlete. In some embodiments, after an athlete relationship is established between the first athlete and the second athlete, at least some of the group activities that have been determined to include the first athlete and the second athlete are presented for either or both athletes so that they can receive/view a history of their group activities and/or any flyby activities that have been determined relative to GPS recorded activities performed by the first athlete and/or GPS recorded activities performed by the second athlete.

In some embodiments, athlete relationships of a first athlete with other athletes can be shared (e.g., in an anonymized form) with a third party social network server such that the athletic relationships can be presented at a social network profile belonging to the first athlete and/or second athlete.

In some embodiments, athlete relationships (e.g., established using a process such as process 600) can be compared to stored data associated with inter-athlete relationships that is derived from other sources or processes (for example, whether or not athletes are connected in an existing “friend” or “following” relationship on a social network).

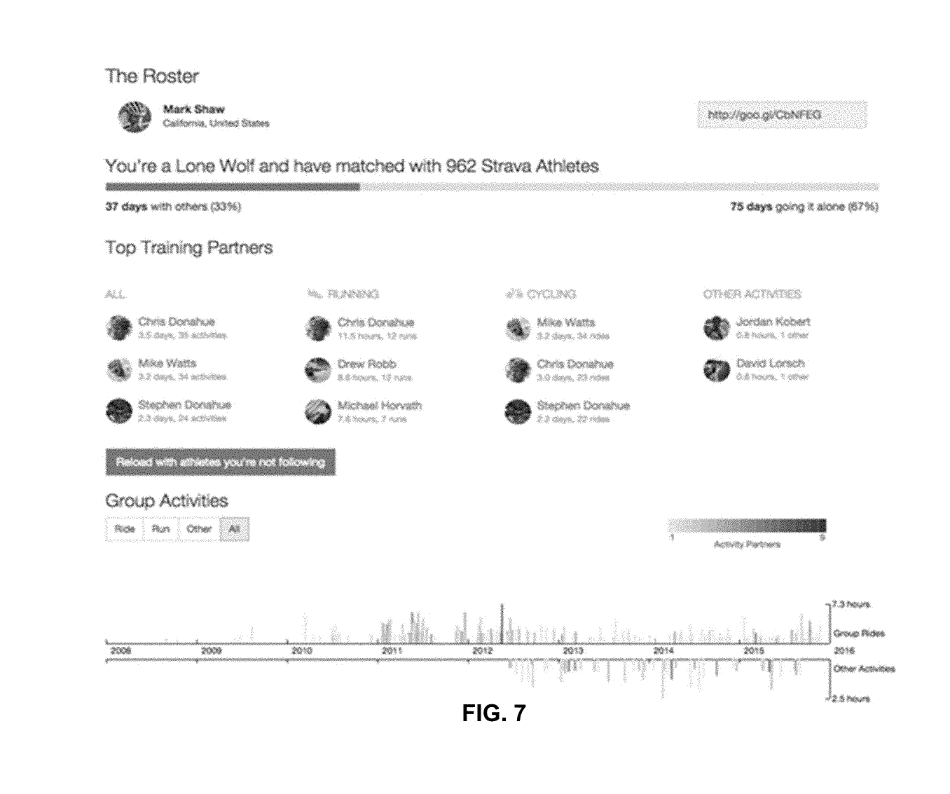

FIG. 7 is an example of a user interface that presents a first athlete's athletic relationships with other athletes. In the example of FIG. 7, the first athlete is “Mark Shaw.” The example user interface presents Mark's athletic relationships (training partners) with other athletes across different types of physical activities (running, cycling), as well as Mark's aggregated shared values with these other athletes (total time and total distance traveled together). The example user interface also presents Mark's lone wolf activities, which are Mark's GPS recorded activities that are not associated with any other athletes and/or determined to be included in group activities with other athletes. The example user interface additionally presents a histogram of Mark's time spent in group activities with respect to different numbers of training partners over time. While not shown in FIG. 7, flyby activities performed by other athletes relative to the GPS recorded activities that have been performed by Mark can also be displayed at the user interface.

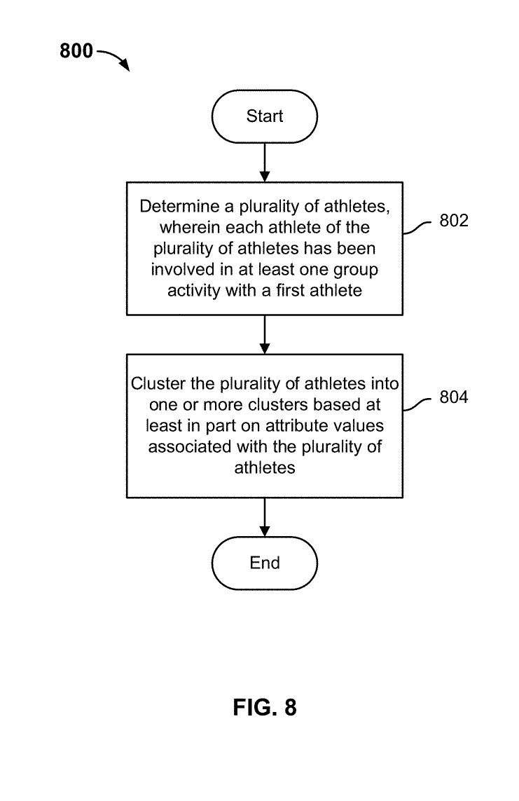

FIG. 8 is a flow diagram showing a process for clustering athletes that have been determined to have participated in one or more group activities with a first athlete in accordance with some embodiments. In some embodiments, process 800 is implemented at a system such as system 100 of FIG. 1.

Process 800 describes determining clusters of athletes that have performed one or more group activities with a first athlete.

At 802, a plurality of athletes is determined, wherein each athlete of the plurality of athletes has been involved in at least one group activity with the first athlete. Various athletes, each of whom has been determined to have participated in at least one group activity as a first athlete are determined.

At 804, the plurality of athletes is clustered based at least in part on attribute values associated with the plurality of athletes. Attributes associated with these athletes, attributes associated with the first athlete, and/or attributes associated with the group activities can be used to cluster the athletes into one or more clusters. For example, the athletes can be clustered multiple times, each time along a different dimension (e.g., time of group activity, activity type of group activity, relationship to the first athlete). In a first example, athletes that have performed group activities with the first athlete in the morning can be clustered into a cluster associated with “morning riders.” In a second example, athletes that frequently compete in competitions can be clustered into a cluster associated with “racers.” Other example clusters may include “commuters”, “swimmers”, “runners”, “multisport athletes” such as “triathletes”, “coworkers.”

Although the foregoing embodiments have been described in some detail for purposes of clarity of understanding, the invention is not limited to the details provided. There are many alternative ways of implementing the invention. The disclosed embodiments are illustrative and not restrictive.

1. A system, comprising:
a processor configured to:
receive a plurality of GPS recorded activities uploaded by a plurality of athletes, wherein the plurality of athletes comprises a first athlete and a second athlete;
receive a request to determine a set of group activities that includes the first athlete and the second athlete;
determine the set of group activities based at least in part on a set of time criteria and a set of space criteria used to compare a first GPS recorded activity associated with the first athlete and a second GPS recorded activity associated with the second athlete; and
output at least some of the set of group activities; and
a memory coupled to the processor and configured to provide instructions to the processor.

2. The system of claim 1, wherein the request to determine the set of group activities that includes the first athlete and the second athlete comprises receipt of a newly uploaded GPS recorded activity from the first athlete or the second athlete.

3. The system of claim 1, wherein to the determine the set of group activities comprises to:
obtain a first set of tiles from the first GPS recorded activity associated with the first athlete; and
obtain a second set of tiles from the second GPS recorded activity associated with the second athlete.

4. The system of claim 1, wherein to the determine the set of group activities comprises to:
obtain a first set of tiles from the first GPS recorded activity associated with the first athlete;
obtain a second set of tiles from the second GPS recorded activity associated with the second athlete; and
determine that the first set of tiles and the second set of tiles share at least one tile or include neighboring tiles that include GPS data points that were recorded within a predetermined window of time of each other.

5. The system of claim 1, wherein to the determine the set of group activities comprises to:
obtain a first set of tiles from the first GPS recorded activity associated with the first athlete;
obtain a second set of tiles from the second GPS recorded activity associated with the second athlete;
determine that the first set of tiles and the second set of tiles share at least one tile or include neighboring tiles that include GPS data points that were recorded within a predetermined window of time of each other; and
determine that the second GPS recorded activity comprises a flyby activity in relation to the first GPS recorded activity.

6. The system of claim 1, wherein to the determine the set of group activities comprises to:
obtain a first set of tiles from the first GPS recorded activity associated with the first athlete;
obtain a second set of tiles from the second GPS recorded activity associated with the second athlete;
determine that the first set of tiles and the second set of tiles share at least one tile or include neighboring tiles that include GPS data points that were recorded within a predetermined window of time of each other;
determine a correlation in space between the first GPS recorded activity and the second GPS recorded activity; and
wherein in the event that the correlation exceeds a predetermined correlation threshold, determine that the first GPS recorded activity and the second GPS recorded activity form a group activity that includes the first athlete and the second athlete.

7. The system of claim 1, wherein the processor is configured to:
determine a set of shared values corresponding to respective ones of the set of group activities;
aggregate the set of shared values to determine an aggregate shared value between the first athlete and the second athlete;
determine whether the aggregated shared value exceeds a predetermined threshold aggregated shared value; and
wherein in the event that the aggregated shared value exceeds the predetermined threshold aggregated shared value, present a prompt associated with suggesting to establish an athletic relationship between the first athlete and the second athlete.

8. The system of claim 1, wherein the processor is configured to:
determine a set of shared values corresponding to respective ones of the set of group activities;
aggregate the set of shared values to determine an aggregate shared value between the first athlete and the second athlete;
determine whether the aggregated shared value exceeds a predetermined threshold aggregated shared value;
wherein in the event that the aggregated shared value exceeds the predetermined threshold aggregated shared value, present a prompt associated with suggesting an athletic relationship between the first athlete and the second athlete; and
send data associated with the athletic relationship between the first athlete and the second athlete to a server associated with a social network service.

9. The system of claim 1, wherein the processor is configured to:
receive a search query; and
determine one or more group activities included in the set of group activities or one or more athletes associated with the set of group activities that match the search query.

10. The system of claim 1, wherein the processor is configured to:
determine a set of athletes other than the first athlete that is associated with the set of group activities; and
cluster the plurality of athletes into one or more clusters based at least in part on attribute values associated with the set of athletes.

11. A method, comprising:
receiving a plurality of GPS recorded activities uploaded by a plurality of athletes, wherein the plurality of athletes comprises a first athlete and a second athlete;
receiving a request to determine a set of group activities that includes the first athlete and the second athlete;
determining the set of group activities based at least in part on a set of time criteria and a set of space criteria used to compare a first GPS recorded activity associated with the first athlete and a second GPS recorded activity associated with the second athlete; and
outputting at least some of the set of group activities.

12. The method of claim 11, wherein the request to determine the set of group activities that includes the first athlete and the second athlete comprises receipt of a newly uploaded GPS recorded activity from the first athlete or the second athlete.

13. The method of claim 11, wherein determining the set of group activities comprises:
obtaining a first set of tiles from the first GPS recorded activity associated with the first athlete; and
obtaining a second set of tiles from the second GPS recorded activity associated with the second athlete.

14. The method of claim 11, wherein determining the set of group activities comprises:
obtaining a first set of tiles from the first GPS recorded activity associated with the first athlete;
obtaining a second set of tiles from the second GPS recorded activity associated with the second athlete; and
determining that the first set of tiles and the second set of tiles share at least one tile or include neighboring tiles that include GPS data points that were recorded within a predetermined window of time of each other.

15. The method of claim 11, wherein determining the set of group activities comprises:
obtaining a first set of tiles from the first GPS recorded activity associated with the first athlete;
obtaining a second set of tiles from the second GPS recorded activity associated with the second athlete;
determining that the first set of tiles and the second set of tiles share at least one tile or include neighboring tiles that include GPS data points that were recorded within a predetermined window of time of each other; and
determining that the second GPS recorded activity comprises a flyby activity in relation to the first GPS recorded activity.

16. The method of claim 11, wherein determining the set of group activities comprises:
obtaining a first set of tiles from the first GPS recorded activity associated with the first athlete;
obtaining a second set of tiles from the second GPS recorded activity associated with the second athlete;
determining that the first set of tiles and the second set of tiles share at least one tile or include neighboring tiles that include GPS data points that were recorded within a predetermined window of time of each other;
determining a correlation in space between the first GPS recorded activity and the second GPS recorded activity; and
wherein in the event that the correlation exceeds a predetermined correlation threshold, determining that the first GPS recorded activity and the second GPS recorded activity form a group activity that includes the first athlete and the second athlete.

17. The method of claim 11, further comprising:
determining a set of shared values corresponding to respective ones of the set of group activities;
aggregating the set of shared values to determine an aggregate shared value between the first athlete and the second athlete;
determining whether the aggregated shared value exceeds a predetermined threshold aggregated shared value; and
wherein in the event that the aggregated shared value exceeds the predetermined threshold aggregated shared value, presenting a prompt associated with suggesting to establish an athletic relationship between the first athlete and the second athlete.

18. The method of claim 11, further comprising:
determining a set of shared values corresponding to respective ones of the set of group activities;
aggregating the set of shared values to determine an aggregate shared value between the first athlete and the second athlete;
determining whether the aggregated shared value exceeds a predetermined threshold aggregated shared value;
wherein in the event that the aggregated shared value exceeds the predetermined threshold aggregated shared value, presenting a prompt associated with suggesting an athletic relationship between the first athlete and the second athlete; and
sending data associated with the athletic relationship between the first athlete and the second athlete to a server associated with a social network service.

19. The method of claim 11, further comprising:
determining a set of athletes other than the first athlete that is associated with the set of group activities; and
clustering the plurality of athletes into one or more clusters based at least in part on attribute values associated with the set of athletes.

20. A computer program product, the computer program product being embodied in a non-transitory computer readable storage medium and comprising computer instructions for:
receiving a plurality of GPS recorded activities uploaded by a plurality of athletes, wherein the plurality of athletes comprises a first athlete and a second athlete;
receiving a request to determine a set of group activities that includes the first athlete and the second athlete;
determining the set of group activities based at least in part on a set of time criteria and a set of space criteria used to compare a first GPS recorded activity associated with the first athlete and a second GPS recorded activity associated with the second athlete; and
outputting at least some of the set of group activities.
